(19) United States
(12) Patent Application Publication
(10) Pub. No.: US 2022/0238192 A1
(43) Pub. Date: Jul. 28, 2022

(54) METHOD FOR CREATING AN INDIVIDUAL GENE PANEL PLAN

(71) Applicant: Siemens Healthcare GmbH, Erlangen (DE)

(72) Inventors: Oliver FRINGS, Erlangen (DE); Maximilian WEISS, Nuernberg (DE); Lisa VALLINES, Weisendorf (DE)

(73) Assignee: Siemens Healthcare GmbH, Erlangen (DE)

(21) Appl. No.: 17/577,509

(22) Filed: Jan. 18, 2022

(30) Foreign Application Priority Data
Jan. 26, 2021 (DE) 102021200650.7

(51) Int. Cl. G16H 10/20; G16H 70/60; G16B 20/00

(52) U.S. Cl. CPC G16H 10/20; G16B 20/00; G16H 70/60

(57) ABSTRACT

A computer-implemented method is for creating an individual gene panel plan. The method includes receiving and/or determining a plurality of clinical trials. In this case, each clinical trial of the plurality of clinical trials includes a molecular genetic inclusion criterion. The molecular genetic inclusion criterion relates in this case to gene information relevant to the respective clinical trial. The method further includes determining, for each clinical trial of the plurality of clinical trials, at least one genomic region to which the gene information of the clinical trial relates. The method further includes creating the gene panel plan based on the genomic regions determined in respect of the plurality of clinical trials. The method further includes providing the gene panel plan.

### PRIORITY STATEMENT

The present application hereby claims priority under 35 U.S.C. § 119 to German patent application number DE 102021200650.7 filed Jan. 26, 2021, the entire contents of which are hereby incorporated herein by reference.

### FIELD

Example embodiments of the invention generally relate to a method for creating an individual gene panel plan; a determination system; a computer program product and a computer-readable storage medium.

### BACKGROUND

A clinical trial is typically conducted in order to test the effectiveness of a therapy and/or a medication on a limited number of participants or patients. Participants in a clinical trial must typically satisfy at least one molecular genetic and/or phenotypic inclusion criterion in order to be able to take part in the clinical trial or to be eligible to participate. In particular, it is assumed as a premise that the therapy or medication tested in the clinical trial has a particularly advantageous effect in the case of a patient that satisfies the at least one molecular genetic and/or phenotypic inclusion criterion. A molecular genetic inclusion criterion may for example comprise information about a molecular change in a particular gene in a patient and/or about a particular expression of a gene in a patient and/or about a gene mutation in a patient. A phenotypic inclusion criterion may for example be an age of a patient and/or a disease of the patient that is to be treated.

For example, a clinical trial may be designed for the treatment of cancer patients. In other words, a therapy or drug for treating a cancer or tumor disease can be tested in the corresponding clinical trial. In particular, the at least one molecular genetic inclusion criterion may then comprise information about a gene mutation that characterizes the tumor treated in the clinical trial. In particular, those patients whose tumor is characterized by the gene mutation can then be included in the clinical trial.

Typically, there are a large number of ongoing clinical trials. Finding a clinical trial for which a particular patient is eligible often involves a great deal of effort. In order to check whether the patient is eligible for a clinical trial, it is necessary to consider whether he or she satisfies the at least one molecular genetic and/or phenotypic inclusion criterion relevant to the clinical trial.

In order to be able to select a clinical trial appropriate for the patient in terms of the molecular genetic inclusion criterion from the large number of ongoing clinical trials, the genes of the patient can be examined or analyzed individually. A check can subsequently be conducted to determine whether the patient satisfies at least one molecular genetic inclusion criterion of at least one ongoing clinical trial. However, analyzing the individual genes is associated with high costs and is extremely time-consuming.

In order to check whether the patient is eligible for a clinical trial out of the large number of ongoing clinical trials in terms of the molecular genetic inclusion criterion, it is alternatively known to analyze a plurality of genes of the patient in a gene panel. In this case it is possible to choose from a plurality of predefined gene panels. A gene panel comprises a fixed plurality of genes and/or genome regions that are examined or analyzed. In this regard, a predefined gene panel typically comprises 30 to 150 genes or genome regions. Selecting these genes or genome regions is often erratic and not therapy-dependent. In particular, the selection of the genes or genome regions on a predefined gene panel is often dependent on a provider of the gene panel. In particular, genes or genome regions that are not encompassed by any molecular genetic inclusion criterion are also frequently examined on a gene panel, while genes or genome regions that are relevant in terms of the molecular genetic inclusion criteria are not examined. In other words, genes or genome regions that are not relevant to any clinical trial are frequently examined in a predefined gene panel, while relevant genes or genome regions are not examined.

### SUMMARY

At least one embodiment of the present invention provides a method which enables an individual gene panel plan to be created for a patient in respect of a plurality of clinical trials.

Embodiments are directed to a method for creating an individual gene panel plan; a determination system for creating an individual gene panel plan; a computer program product and a computer-readable storage medium. Advantageous developments are set forth in the claims and in the following description.

Embodiments are described below both in relation to the claimed devices and in relation to the claimed method.

Features, advantages or alternative embodiments mentioned in this context are equally to be applied also to the other claimed subject matters, and vice versa. In other words, the object-related claims (which are directed for example to a device) can also be developed by way of the features that are described or claimed in connection with a method. The corresponding functional features of the method are in this case embodied by way of corresponding object-related modules.

At least one embodiment of the invention relates to a computer-implemented method for creating an individual gene panel plan. The method comprises a method step of receiving and/or determining a plurality of clinical trials. Each clinical trial of the plurality of clinical trials in this case comprises a molecular genetic inclusion criterion. The molecular genetic inclusion criterion relates in this case to gene information relevant to the respective clinical trial. The method further comprises a method step of determining, for each clinical trial of the plurality of clinical trials, at least one genomic region to which the gene information of the clinical trial relates. The method further comprises a method step of creating the gene panel plan based on the genomic regions determined in respect of the plurality of clinical trials. The method further comprises a method step of providing the gene panel plan.

At least one embodiment of the invention optionally relates to a computer-implemented training method for providing a trained function. The training method comprises a method step of receiving an available set of clinical trials and patient data of a patient. The training method further comprises a method step of receiving clinical trials relevant to the patient. The clinical trials relevant to the patient and the available set of clinical trials as well as the patient data are interrelated in this case. The available set of clinical trials in this case comprises the clinical trials relevant to the patient. The training method further comprises a method step of training a function based on the available set of clinical trials, the patient data and the clinical trials relevant to the patient. The training method further comprises a method step of providing the trained function.

At least one embodiment of the invention further relates to a determination system for creating an individual gene panel plan comprising an interface and a computing unit. In this case the interface and/or the computing unit are/is embodied to receive and/or determine a plurality of clinical trials. Each clinical trial of the plurality of clinical trials in this case comprises a molecular genetic inclusion criterion. The molecular genetic inclusion criterion in this case relates to gene information relevant to the respective clinical trial. In this case the computing unit is further embodied for determining, for each clinical trial of the plurality of clinical trials, at least one genomic region to which the gene information of the clinical trial relates. In this case the computing unit is further embodied to create a gene panel plan based on the genomic regions determined in respect of the plurality of clinical trials. In this case the interface is further embodied to provide the gene panel plan.

At least one embodiment of the invention also relates to a computer program product comprising a computer program, as well as to a computer-readable medium. A largely software-based implementation has the advantage that determination systems already used previously in the prior art can also be easily upgraded via a software update in order to operate in the manner described. In addition to the computer program, such a computer program product may, where applicable, comprise additional constituent parts such as e.g. a set of documentation and/or additional components, as well as hardware components, such as e.g. hardware keys (dongles, etc.) to enable use of the software.

In particular, at least one embodiment of the invention also relates to a computer program product comprising a computer program which can be loaded directly into a memory of a determination system and having program sections for performing all steps of the above-described method for creating an individual gene panel plan and its embodiments when the program sections are executed by the determination system.

In particular, at least one embodiment of the invention relates to a computer-readable storage medium on which are stored program sections that can be read and executed by a determination system in order to perform all steps of at least one embodiment of the above-described method for creating an individual gene panel plan and its embodiments when the program sections are executed by the determination system.

At least one embodiment of the invention optionally relates to a training system for providing a trained function comprising a training interface and a training computing unit. The training interface is embodied to receive an available set of clinical trials and patient data of a patient. The training interface is further embodied to receive clinical trials relevant to the patient. In this case the clinical trials relevant to the patient and the available set of clinical trials as well as the patient data are interrelated. In this case the available set of clinical trials comprises the clinical trials relevant to the patient. The training computing unit is embodied to train a function based on the available set of clinical trials, the patient data and the clinical trials relevant to the patient. The training interface is further embodied to provide the trained function.

At least one embodiment of the invention optionally relates also to a computer program product comprising a computer program, as well as to a computer-readable medium. A largely software-based implementation has the advantage that training systems already used previously can also be easily upgraded via a software update in order to operate in the manner described. In addition to the computer program, such a computer program product may, where applicable, comprise additional constituent parts such as e.g. a set of documentation and/or additional components, as well as hardware components, such as e.g. hardware keys (dongles, etc.) to enable use of the software.

In particular, at least one embodiment of the invention optionally relates also to a computer program product comprising a computer program which can be loaded directly into a memory of a training system and having program sections for performing all steps of at least one embodiment of the above-described training method for providing a trained function and its aspects when the program sections are executed by the training system.

In particular, at least one embodiment of the invention optionally relates to a computer-readable storage medium on which are stored program sections that can be read and executed by a training system in order to perform all steps of at least one embodiment of the above-described training method for creating an individual gene panel plan and its aspects when the program sections are executed by the training system.

At least one embodiment of the invention relates to a computer-implemented method for creating an individual gene panel plan, the method comprising:

at least one of receiving and determining a plurality of clinical trials, each respective clinical trial of the plurality of clinical trials including a molecular genetic inclusion criterion, wherein the molecular genetic inclusion criterion relates to gene information relevant to the respective clinical trial; and

for each respective clinical trial of the plurality of clinical trials,
determining at least one genomic region to which the gene information of the clinical trial relates,
creating the gene panel plan based on the one genomic regions determined in respect of the plurality of clinical trials, and
providing the gene panel plan created.

At least one embodiment of the invention relates to a determination system for creating an individual gene panel plan, comprising:

an interface; and

a computing unit,

wherein at least one of the interface and the computing unit is embodied to at least one of receive and determine a plurality of clinical trials, each respective clinical trial of the plurality of clinical trials including a molecular genetic inclusion criterion, wherein the molecular genetic inclusion criterion relates to gene information relevant to the respective clinical trial;

wherein the computing unit is further embodied to determine, for each respective clinical trial of the plurality of clinical trials, at least one genomic region to which the gene information of the clinical trial relates,

wherein the computing unit is further embodied to create a gene panel plan based on the genomic regions determined in respect of the plurality of clinical trials, and

wherein the interface is further embodied to provide the gene panel plan created.

At least one embodiment of the invention relates to a non-transitory computer program product storing a computer program, directly loadable into a memory of a determination system and including program sections for performing the method of t least one embodiment of the invention when the program sections are executed by the determination system.

At least one embodiment of the invention relates to a non-transitory computer-readable storage medium storing program sections, readable and executable by a determination system, to perform the method of at least one embodiment of the invention when the program sections are executed by the determination system.

### BRIEF DESCRIPTION OF THE DRAWINGS

The above-described characteristics, features and advantages of this invention will become clearer and more readily understandable in connection with the following figures and their descriptions. At the same time, the figures and descriptions are not intended to limit the invention and its embodiments in any way.

Like components are labeled with corresponding reference signs in different figures. The figures are generally not to scale.

In the Figures:

FIG. 1 shows a first example embodiment of a method for creating an individual gene panel plan,

FIG. 2 shows a second example embodiment of a method for creating an individual gene panel plan,

FIG. 3 shows a first example embodiment of a method step for determining clinical trials relevant to a patient,

FIG. 4 shows a second example embodiment of a method step for determining clinical trials relevant to a patient,

FIG. 5 shows a third example embodiment of a method for creating an individual gene panel plan,

FIG. 6 shows a determination system for creating an individual gene panel plan,

FIG. 7 shows a training system for providing a trained function.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description.

However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

least one embodiment of the invention relates to a computer-implemented method for creating an individual gene panel plan. The method comprises a method step of receiving and/or determining a plurality of clinical trials. Each clinical trial of the plurality of clinical trials in this case comprises a molecular genetic inclusion criterion. The molecular genetic inclusion criterion relates in this case to gene information relevant to the respective clinical trial. The method further comprises a method step of determining, for each clinical trial of the plurality of clinical trials, at least one genomic region to which the gene information of the clinical trial relates. The method further comprises a method step of creating the gene panel plan based on the genomic regions determined in respect of the plurality of clinical trials. The method further comprises a method step of providing the gene panel plan.

In the method step of receiving and/or determining the plurality of clinical trials, the plurality of clinical trials is received and/or determined. In particular, the plurality of clinical trials comprises those trials for which a check is to be conducted to determine whether a patient is eligible for at least one of the clinical trials. In other words, a check is to be conducted for the plurality of clinical trials in order to determine whether the patient is eligible to take part in at least one of the clinical trials. In particular, each clinical trial of the plurality of clinical trials is an ongoing clinical trial. An eligible patient or participant can be registered for an ongoing clinical trial.

The plurality of clinical trials may in particular be received by a database. In this case the database may be for example one of the following databases: ClinicalTrials.gov (https://clinicaltrials.gov/), International Clinical Trials Registry Platform (acronym: ICTRP) (https://www.who.int/clinical-trials-registry-platform), Deutsches Register Klinischer Studien (acronym: DRKS; German Clinical Trials Register) (https://www.drks.de/drks_web/) and/or EU Clinical Trials Register (https://www.clinicaltrialsregister.eu/).

Each clinical trial of the plurality of clinical trials can in this case be assigned a unique identification number or a unique indicator (acronym: ID). In other words, each clinical trial of the plurality of clinical trials can be uniquely identifiable via an ID. In particular, each clinical trial of the plurality of clinical trials may comprise the corresponding ID.

In a clinical trial, in particular an effectiveness and/or a compatibility of a therapy and/or a medicine is tested on a limited number of patients. In particular, a clinical trial can be designed for certain patients possessing certain characteristics. In other words, only patients that possess at least some of the particular characteristics predefined by the clinical trial may take part in the clinical trial. The characteristics may in this case be for example genetic, molecular genetic, phenotypic characteristics, etc. Such a predefined characteristic may be referred to in particular as an inclusion criterion.

Each of the clinical trials of the plurality of clinical trials in this case comprises a molecular genetic inclusion criterion. In this regard, the molecular genetic inclusion criterion relates to gene information relevant to the respective clinical trial. In particular, a condition for a participation of a patient in the corresponding clinical trial is defined by the molecular genetic inclusion criterion. Via the gene information, the molecular genetic inclusion criterion in particular defines a condition for participation of the patient in the corresponding clinical trial in respect of the genome of the patient. Relevant means in this context that the gene information comprises information relevant to participation in the corresponding clinical trial. The gene information may in this case comprise in particular a genetic prerequisite or condition for participation in the clinical trial. The gene information may in this case directly define the genetic prerequisite. For example, the gene information can define at least one gene and/or at least one region of a genome that must have a defined expression in order to take part in the clinical trial. Alternatively, the gene information can indirectly define the genetic prerequisite for taking part in the clinical trial. For example, the gene information can comprise a name or designation for an expression of at least one gene and/or at least one region of a genome which is a condition for participation in the clinical trial. Alternatively or in addition, the gene information can comprise a designation for a disease that is caused by a particular genetic expression which embodies the molecular genetic inclusion criterion. For example, the designation “BRCA” can point to a change in a gene relevant to breast cancer or the designation “hemophilia”, for example, to a change in the expression of a gene on the X chromosome.

In particular, an available set of clinical trials can be filtered in the step of determining the plurality of clinical trials in such a way that only clinical trials of the available set of clinical trials that comprise a molecular genetic inclusion criterion are part of the plurality of clinical trials. In this case the plurality of clinical trials is a subset of the available set of clinical trials. In other words, the plurality of clinical trials may comprise a selection of the clinical trials that include a molecular genetic inclusion criterion. The available set of clinical trials can in particular comprise ongoing clinical trials. The available set of clinical trials can be provided for example by at least one of the above-cited databases.

In the method step of determining the genomic region to which the gene information of the clinical trial relates, the genomic region is determined for each trial based upon the gene information. The genomic region defines in particular an area or a region on a genome of the patient which, according to the gene information, must exhibit a specific expression in order to be eligible for participation in the corresponding clinical trial. The genome may be in particular a human genome. Alternatively, the genome can be an animal genome. The genomic region can in this case be copied in particular directly from the gene information. In particular, the genomic region can be copied directly from the gene information when the gene information directly defines the genetic prerequisite. Alternatively, the genomic region can be determined based on the gene information when the gene information indirectly defines the genetic prerequisite. In particular, the genomic region can then be derived from the gene information with the aid of specialist knowledge and/or general specifications and/or a database.

In the method step of creating the gene panel plan, the individual gene panel plan is determined based on the genomic regions determined in respect of the plurality of clinical trials. The gene panel plan in this case comprises in particular all the genomic regions of the plurality of clinical trials. The gene panel plan defines in particular a totality of all the genomic regions of the plurality of clinical trials. In particular, a unification of all the genomic regions of the plurality of clinical trials can be defined in the gene panel plan. In particular, the gene panel plan describes which genomic regions of the patient are to be examined or analyzed in order to establish whether the patient is eligible to take part in at least one of the clinical trials of the plurality of clinical trials. In particular, the gene panel plan comprises only the genomic regions that are relevant in order to check the eligibility of the patient for the clinical trials of the plurality of clinical trials. In other words, the gene panel plan includes no regions of the genome that are irrelevant for a participation in the plurality of the clinical trials. In particular, a gene panel can be created based upon the gene panel plan. In other words, the gene panel plan defines a gene panel. In the gene panel, the genomic regions included in the gene panel plan can be analyzed. In particular, the gene panel plan is determined individually for the patient. In particular, the gene panel plan is specific to the plurality of clinical trials.

In the method step of providing the gene panel plan, the gene panel plan can be provided to a user. The user may be in particular a physician who decides on the participation of the patient in one of the clinical trials of the plurality of clinical trials. Alternatively or in addition, the user can also be the patient. Alternatively or in addition, the user can be a medical assistant. Alternatively or in addition, the user can be a provider that creates and analyzes a gene panel based upon the gene panel plan. The gene panel plan can be provided via a user interface. In particular, the gene panel plan can be displayed to the user via a monitor. Alternatively or in addition, the gene panel plan can be provided in a Browser Extensible Data format (.bed document or .bed file). In particular, the gene panel plan can be transmitted to the provider electronically in the form of a .bed document. In particular, the electronic transmission can be a transmission via email and/or via a cloud system and/or via a database, etc.

The inventors have recognized that an individual gene panel plan can be created by determining the genomic regions for the plurality of clinical trials. In particular, the gene panel plan can be produced in respect of the plurality of clinical trials. In other words, the gene panel plan can be created specifically for the plurality of clinical trials. The inventors have recognized that in this way it is possible to produce a gene panel plan via which it can be checked individually for a patient whether he or she is eligible for at least one clinical trial of the plurality of clinical trials or whether he or she satisfies a molecular genetic inclusion criterion of at least one clinical trial. The inventors have recognized that based upon the gene panel plan a gene panel can be produced which analyzes precisely the genomic regions relevant to the plurality of clinical trials. In particular, time can be saved in this way since only the genomic regions relevant to the participation in at least one clinical trial of the plurality of clinical trials are analyzed. Costs can also be saved in this way since the analysis is targeted only at the relevant genomic regions in the gene panel based on the gene panel plan that are relevant for participation in at least one of the clinical trials.

According to an embodiment of the invention, the gene information of a clinical trial relates to at least one region of a genome relevant to the clinical trial.

In other words, the gene information provides information about which region of a genome is relevant to the participation of the patient in the corresponding trial. In particular, the at least one region of a genome whose expression is relevant to a decision on the participation of the patient in the clinical trial is defined via the gene information. In particular, the relevant region describes the region of a gene on a genome that must possess a specific expression in order to satisfy the molecular genetic inclusion criterion. In particular, the gene information can define more than one relevant region. In particular, “relevant” in this context means that an expression of the relevant region of a genome is at least also included as a contributory factor when deciding on the eligibility of the patient for the corresponding clinical trial. In particular, the relevant region of a genome can comprise the entire gene. In other words, the gene information can relate to at least one entire gene.

The inventors have recognized that the at least one region of a genome relevant to the clinical trial can be defined via the gene information. In particular, the inventors have recognized that via the gene information it is possible to restrict the gene panel plan to the regions of the plurality of clinical trials defined or provided in the gene information. In particular, the inventors have recognized that the clinical trials that comprise a molecular genetic inclusion criterion define the at least one relevant region of a genome in the corresponding gene information.

According to a further embodiment of the invention, the at least one relevant region of the genome contains a gene mutation relevant to the clinical trial.

A gene mutation means that there has been a change in the corresponding region of the genome compared to a standard. The standard may be for example an expression of the region of the genome in a majority of a population. In other words, a gene mutation involves a change in the genetic makeup in the corresponding region of the genome. In particular, the gene mutation may be a gene defect. In particular, the gene defect is a gene mutation that has a negative impact on the gene or on the genome. For example, a gene defect can lead to an uninhibited growth of a cell containing the gene.

In particular, the gene information relates to the at least one relevant region of the genome which contains the gene mutation. In particular, the gene information can define where or on which gene or in which region of the genome the gene mutation is located.

In particular, the therapy tested in the corresponding trial or the medication tested in the corresponding trial may have a particularly advantageous effect in the case of a patient carrying the corresponding gene mutation.

The inventors have recognized that a gene mutation is frequently a molecular genetic inclusion criterion for participation in a clinical trial. The inventors have recognized that the gene information may include information about the gene mutation. The inventors have recognized that an expression of a region of a genome relevant to a clinical trial may be a gene mutation.

According to a further embodiment of the invention, the gene information comprises a name of the region of the genome exhibiting the gene mutation and/or a name for the gene mutation.

The name of the region containing the gene mutation may be in particular a name of the mutated gene. A list of all possible names of genes is provided for example in the database of the HUGO Gene Nomenclature Committee (acronym: HGNC) (https://www.genenames.org/).

In particular, it is possible, via the name of the region of the genome containing the gene mutation and/or the name of the gene mutation, to infer in which region of the genome the gene mutation is located.

For example, a Kirsten Rat Sarcoma Mutation (KRAS mutation) designates a mutation of the KRAS gene. The KRAS gene is responsible for the production of a K-ras protein. The K-ras protein is responsible for the growth process of a cell. A mutation of the KRAS gene can lead to a disrupted cell growth. It is known in which genomic region the KRAS gene is arranged. In particular, the gene information may therefore comprise the name of the mutation (KRAS mutation) and/or the name of the mutated gene (KRAS gene). In particular, the corresponding genomic region can be derived therefrom in the step of determining the genomic region.

Alternatively or in addition, the gene information can comprise a name of a mutated region of a genome, for example EGFR exon 21 or EGRF p.L858R. In particular, the genomic region can be determined more precisely therefrom since only the corresponding region of the genome can be determined as the genomic region at the site of the gene mutation. For example, the name “EGFR exon 21” describes the entire Exon 21 of the EGFR gene, whereas the name “EGRF p.L858R” indicates a point mutation of the 858 amino acid of the EGFR gene. Alternatively, the gene information can comprise the name of the corresponding gene mutation, for example EGFR exon 21 deletion. In particular, the region of the gene mutation on the genome can be derived from the name of the gene mutation.

In particular, the name of the gene mutation can be an abstract name for a disorder resulting from the gene mutation. For example, the condition “hemophilia” can point to a mutation of a certain gene on the X chromosome. Such an abstract name can be “Her2 pos”, for example, which points to a mutation in breast cancer.

The inventors have recognized that the genomic region relevant to the clinical trial can be derived based upon the name of the gene mutation and/or the name of the region of the genome having the gene mutation. The inventors have recognized that the gene information may therefore comprise in particular the name of the gene mutation and/or the name of the region containing the gene mutation. The inventors have recognized that this information is frequently provided in a clinical trial. The inventors have recognized that existing information can therefore be used.

According to a further embodiment of the invention, the genomic region comprises coordinates of the at least one region of the genome relevant to the clinical trial.

In particular, the coordinates define a position of the relevant region on the genome. In particular, the coordinates can define a relevant region on the genome. In particular, a subregion of a gene can be defined as a relevant region in this way. The coordinates can in particular be read out directly from the gene information when the gene information comprises the coordinates of the relevant region.

Alternatively, the coordinates can be derived from the name of the relevant region. In this case the name is included in the gene information. The coordinates can be derived from the name via a database. For example, the ENSEMBL database (https://grch37.ensembl.org/index.html) and/or the NCBI database (https://www.ncbi.nlm.nih.gov/) can be used in order to derive the coordinates from the name. In particular, the coordinates can also be derived from the name of the gene mutation in this way. Alternatively, the name of the relevant region can be derived in turn from the name of the gene mutation.

The inventors have recognized that the coordinates simplify creation of the gene panel plan. In particular, the genomic regions in the gene panel plan that are to be analyzed or examined can be specified precisely via the coordinates. The inventors have recognized that in this way it is possible to avoid analyzing genomic regions in the gene panel based on the gene panel plan that are irrelevant or wrong for the plurality of clinical trials.

According to a further embodiment of the invention, the at least one genomic region additionally comprises a buffer zone around the coordinates of the at least one relevant region of the genome.

In other words, the genomic region comprises a region that is greater than the relevant region itself by a buffer zone. In this case the buffer zone can be in particular equal in size in all directions from the viewpoint of the relevant region. The buffer zone in this case directly adjoins the relevant region. In particular, the buffer zone can for example comprise at least one neighboring gene to the gene which comprises the relevant region. Alternatively, the buffer zone can be embodied in such a way that the genomic region comprises the entire gene, even if the relevant region comprises only a subregion of the gene. In other words, the buffer zone can be embodied in such a way that the genomic region always comprises an entire or complete gene and/or the neighboring genes to the gene encompassed by the relevant region.

The inventors have recognized that via the buffer zone it can be ensured that even in the event of possible inaccuracies in the creation of the gene panel based on the provided gene panel plan, the relevant region is located within the actually analyzed genomic region. In other words, it can be ensured via the buffer zone that the relevant region is located within the actually analyzed genomic region, even if the actually analyzed genomic region is different from the genomic region that is included or defined in the gene panel plan.

According to a further embodiment of the invention, the method step of determining the plurality of clinical trials comprises a method step of determining clinical trials relevant to a patient from an available set of clinical trials by filtering the available set of clinical trials. In this case the plurality of clinical trials comprises the clinical trials relevant to the patient.

The available set of clinical trials can in particular be a set of clinical trials in a database. A database of the type may be for example one of the following databases: https://clinicaltrials.gov/, https://www.who.int/clinical-trials-registry-platform, https://www.drks.de/drks_web/and/or https://www.clinicaltrialsregister.eu/. The clinical trials of the available set of clinical trials may be in particular ongoing clinical trials.

The patient is in particular the patient for whom it is to be checked whether he or she is eligible to take part in at least one clinical trial of the plurality of clinical trials.

In the method step of determining trials relevant to a patient, those trials that are relevant to the patient are selected from the available set of clinical trials. In other words, those clinical trials for which an eligibility of the patient can be ruled out directly based upon simple characteristics are filtered out from the set of clinical trials. In particular, the plurality of clinical trials comprises those trials for which an eligibility of the patient cannot be directly ruled out. In particular, the plurality of clinical trials may be equal to the trials relevant to the patient. In other words, the plurality of clinical trials may comprise only the trials relevant to the patient. In particular, the available set of clinical trials may be equal to the plurality of clinical trials. In other words, the available set of clinical trials and the plurality of clinical trials may include the same clinical trials.

The inventors have recognized that a number of clinical trials in the plurality of clinical trials can be minimized via the filtering. In this way it is possible to speed up the process of determining the genomic regions for all clinical trials of the plurality of clinical trials. In other words, it can be ensured via the filtering that no genomic regions are included in the gene panel plan for the assigned clinical trials of which the patient was ineligible from the outset. The inventors have recognized that what can be achieved in this way is that the gene panel plan defines the smallest possible gene panel. In particular, it can be prevented in this way that too many genomic regions are included in the gene panel plan which make a gene panel based thereon unnecessarily large. In particular, time and costs can be saved in this way.

According to a further embodiment of the invention, at least one clinical trial of the plurality of clinical trials comprises at least one phenotypic inclusion criterion. In this case the clinical trial is designed for treating a disease affecting trial participants. The phenotypic inclusion criterion in this case comprises at least one of the following criteria: an age of the trial participants, a place of residence of the trial participants, the disease affecting the trial participants, a stage of the disease affecting the trial participants. In this case, determining clinical trials relevant to a patient is based on the at least one phenotypic inclusion criterion.

The treatment of the disease in the clinical trial can be effected in particular via a therapy and/or via a medication. The trial participants are in particular patients taking part in the trial. A clinical trial can be designed in particular for a particular group of patients. In particular, the trial participants are then patients from this particular group. At least one common characteristic of this group can be specified in particular via the phenotypic inclusion criterion. In particular, a specification or condition that the patient must satisfy in order to be allowed to take part in the trial can be set based upon the phenotypic inclusion criterion. In particular, a clinical trial may be relevant to the patient when he or she satisfies at least one phenotypic inclusion criterion of the clinical trial.

In particular, more than one clinical trial of the plurality of clinical trials may in each case comprise a phenotypic inclusion criterion. In particular, each clinical trial of the plurality of clinical trials may comprise a phenotypic inclusion criterion. In particular, the clinical trials of the available set of clinical trials that do not comprise a phenotypic inclusion criterion may be included in the plurality of clinical trials. Alternatively, the clinical trials may not be taken into consideration during the filtering process and consequently may not be included in the plurality of clinical trials.

The age of the trial participants specifies in particular an age that the patient is required to be in order to be eligible for the clinical trial. In particular, the age may comprise an age range. For example, the age may limit the trial participants to a group aged between 18 and 60 years old or to a group aged more than 60 years old. Various other variants for limiting the age of the trial participants based upon age are conceivable.

The place of residence of the trial participants specifies in particular where the patient should live in order to be eligible to take part in the clinical trial. For example, a clinical trial may be approved only for a certain country, Germany for example. In particular, the patient should then live in this country in order to be eligible for the trial. In particular, all the trial participants should then live in this particular country. Alternatively, it can be ensured via the place of residence that only patients take part in the clinical trial who can easily get from their place of residence to a particular clinic and/or a particular practice and/or a particular laboratory for follow-up examinations and/or checkups and/or examinations to prepare for the clinical trial and/or for treatment, etc. In other words, the place of residence of the trial participants defines a group of patients that live within a particular vicinity or a particular geographical region.

The disease affecting the trial participants specifies in particular for treating which disease the clinical trial is designed. In particular, the clinical trial may be specific to this disease. In particular, the disease defines a group of patients that suffer from this disease. In particular, the patient is eligible to take part in this clinical trial if he or she has the disease according to the phenotypic inclusion criterion.

The stage of the disease specifies in which stage the disease of the patient should be in order for he or she to be eligible to take part in the clinical trial. In particular, the stage of the disease may be a degree of the disease. In particular, a clinical trial may be designed for example only for the treatment of very far advanced diseases, i.e. for diseases in a late stage or at a high degree. Alternatively, the clinical trial may be designed for the treatment of diseases in an early stage or at a low degree.

In particular, a phenotypic inclusion criterion may comprise further criteria, for example a gender of the trial participants and/or a preexisting condition of the trial participants and/or a previous treatment of the trial participants, etc.

In particular, the filtering in the method step of determining clinical trials relevant to a patient may be based on the at least one phenotypic inclusion criterion. During the filtering of the available set of clinical trials, it can be checked whether the patient satisfies the at least one phenotypic inclusion criterion. In particular, it can be checked whether the age of the patient corresponds to the age of the trial participants and/or whether the place of residence of the patient is located within the geographical region of the places of residence of the trial participants and/or whether the disease of the patient coincides with the disease specific to the participation and/or whether the disease of the patient is in the stage of the disease of the trial participants. In particular, a clinical trial from the available set of clinical trials can be included in the plurality of clinical trials if the patient satisfies at least one phenotypic inclusion criterion of the corresponding clinical trial. In particular, a clinical trial from the available set of clinical trials can be included in the plurality of clinical trials if the patient satisfies all the phenotypic inclusion criteria of the corresponding clinical trial.

The inventors have recognized that via a filtering process based on the phenotypic inclusion criterion it is easily possible to determine those clinical trials from the available set of clinical trials for which the patient is eligible based upon the at least one phenotypic inclusion criterion. In particular, the filtering based on the phenotypic inclusion criterion can be performed quickly and without gathering additional data since the information concerning the patient is typically already available. Accordingly, no additional investment of time or costs is necessary for filtering the available set of clinical trials for trials relevant to the patient.

According to a further embodiment of the invention, the method step of determining the clinical trials relevant to a patient comprises a method step of receiving patient data of the patient. In this case the filtering is based on a synchronizing of the phenotypic inclusion criterion and the patient data.

In particular, the patient data may comprise information about the patient that is required in order to check whether the patient satisfies the at least one phenotypic inclusion criterion. In particular, the patient data may comprise information about the age of the patient and/or about the place of residence of the patient and/or about the disease affecting the patient and/or about the stage of the disease of the patient. In particular, the patient data may be received in the form of a patient's electronic health record. Alternatively or in addition, the patient data may be provided by the patient and/or a treating physician. In other words, the patient data may be received in the form of a user input.

The inventors have recognized that the data required for filtering based on the phenotypic inclusion criterion is typically already stored in the patient data of the patient. The inventors have recognized that the filtering process can therefore be carried out without any additional outlay in terms of time and costs.

According to a further embodiment of the invention, the method step of determining clinical trials relevant to a patient from an available set of clinical trials further comprises the following method steps: receiving patient data of the patient, applying a trained function to the available set of clinical trials and the patient data, a relevance parameter being determined for each clinical trial of the available set of clinical trials, and determining the clinical trials relevant to the patient based on the relevance parameter.

In the method step of receiving patient data, the patient data of the patient is received. In particular, the patient data is received for the patient for whom the relevant clinical trials are to be determined. The patient data may comprise in particular information about an age, a place of residence, a disease to be treated, a stage of the disease to be treated and/or a preexisting condition. In particular, the patient data may have been gathered in the course of a diagnosis and/or a treatment of the disease and/or a treatment of a preexisting condition. In particular, the patient data may be stored in a patient's electronic health record. In particular, the patient data may be provided by the user.

In the method step of applying the trained function, the trained function is applied to the available set of clinical trials and the patient data. In the process, a relevance parameter is generated or determined for each clinical trial. The relevance parameter may specify in particular a relevance of the corresponding clinical trial to the patient. The relevance parameter may specify in particular how highly the patient is eligible for taking part in the corresponding clinical trial. For example, a relevance parameter of “0” can mean that the patient is not eligible and a relevance parameter of “3” can mean that the patient is very highly eligible. Gradations can be mapped using the relevance parameters “1” and “2”. Alternative gradations of the relevance parameter are possible. The trained function may in particular determine the relevance parameter based on the at least one phenotypic inclusion criterion and the patient data. Alternatively or in addition, the trained function may determine the relevance parameter based on the trial participants already assigned to the clinical trial. In particular, the characteristics of the already assigned trial participants, such as, for example, age, place of residence, disease, stage of the disease, may be taken into account when determining the relevance parameter.

Generally, a trained function simulates cognitive functions that human beings associate with human thinking. In particular, via training based on training data, the trained function is able to adapt to new circumstances as well as to detect and extrapolate patterns.

Generally, parameters of a trained function can be adapted via training sessions. In particular, supervised training, semi-supervised training, unsupervised training, reinforcement learning and/or active learning can be used for this purpose. In addition, representation learning (an alternative term is “feature learning”) can also be used. In particular, the parameters of the trained function can be adapted iteratively via multiple training steps.

In particular, a trained function may comprise a neural network, a support vector machine (SVM), a random tree or a decision tree and/or a Bayesian network, and/or the trained function can be based on k-means clustering, Q-learning, genetic algorithms and/or association rules. In particular, a trained function may comprise a combination of multiple uncorrelated decision trees or an ensemble composed of decision trees (random forest). In particular, the trained function may be determined via XGBoosting (eXtreme Gradient Boosting). In particular, a neural network may be a deep neural network, a convolutional neural network or a convolutional deep neural network. In addition, a neural network may be an adversarial network, a deep adversarial network and/or a generative adversarial network. In particular, a neural network may be a recurrent neural network. In particular, a recurrent neural network may be a network with long short-term memory (LSTM), in particular a gated recurrent unit (GRU). In particular, a trained function may comprise a combination of the described approaches. In particular, the approaches described here for a trained function are called the network architecture of the trained function.

In the method step of determining clinical trials relevant to the patient, the clinical trials relevant to the patient are determined based on the relevance parameter. In particular, the relevance parameter is analyzed for each clinical trial of the available set of clinical trials and the clinical trial is classified as relevant or not relevant based on the relevance parameter. The clinical trials relevant to the patient then comprise all the clinical trials that have been classified as relevant. For example, all the clinical trials for which a relevance parameter of “2” or “3” has been determined are classified as relevant and all the clinical trials for which a relevance parameter of “0” or “1” has been determined are classified as not relevant. Other classifications are possible for other gradations of the relevance parameter.

In particular, the filtering of the available set of clinical trials is based on the application of the trained function and the determination of clinical trials relevant to the patient based on the relevance parameter.

The inventors have recognized that with the aid of the trained function it is possible to classify the clinical trials of the available set of clinical trials based upon the relevance parameter in respect of their relevance to the patient. In this way, the trials relevant to the patient can be determined automatically from the available set of clinical trials. The inventors have also recognized that the available data, in particular the patient data, can be used as input data for the trained function. The inventors have recognized that no further, potentially time-consuming and/or cost-intensive, data acquisition is therefore necessary.

According to a further embodiment of the invention, the method step of creating the gene panel plan comprises a method step of combining the genomic regions of the plurality of clinical trials to form at least one combined genomic region, the gene panel plan comprising the at least one combined genomic region.

In particular, the combined genomic region comprises all genomic regions of the plurality of clinical trials. In particular, the gene panel plan may comprise more than one combined genomic region. In particular, the gene panel plan may comprise more than one combined genomic region when the genomic regions are not directly adjacent to one another or do not overlap. In other words, the gene panel plan may comprise more than one combined genomic region when an area or a region between two genomic regions is not encompassed by at least one third genomic region.

The inventors have recognized that via the combining step it can be ensured that all the genomic regions of the plurality of clinical trials are encompassed by the gene panel plan.

According to a further embodiment of the invention, the at least one combined genomic region comprises a unification of all the genomic regions of the plurality of clinical trials.

In other words, the at least one combined genomic region forms a mathematical union of all the genomic regions of the plurality of clinical trials. In particular, the at least one combined genomic region comprises overlapping regions of multiple genomic regions once only. In particular, the at least one combined genomic region comprises no areas or regions on the genome that are not encompassed by any of the genomic regions. In other words, the at least one combined genomic region comprises no region on the genome that is not encompassed by at least one genomic region of the plurality of clinical trials. In particular, the gene panel plan may comprise more than one combined genomic region.

The inventors have established that as a result of the mathematical union of the genomic regions, the at least one combined genomic region comprises the genomic regions in the most compressed manner possible. The inventors have recognized that in this way the gene panel plan is maximally clearly organized. In particular, it can be ensured in this way that the gene panel plan does not comprise overlapping regions of two genomic regions twice. The inventors have recognized that in this way it is possible to prevent the gene panel based on the gene panel plan from analyzing a genomic region or a part of a genomic region more than once. Time and costs can be saved in this way.

According to a further embodiment of the invention, at least one clinical trial of the plurality of clinical trials is designed for a treatment of a tumor disease.

In particular, an effectiveness of a therapy and/or a medication for a tumor disease is tested on a limited number of patients via the at least one clinical trial. In particular, the phenotypic inclusion criterion can then comprise a particular type of the tumor disease, for example a lung carcinoma, a breast carcinoma, a prostate carcinoma, a liver carcinoma or a pancreatic carcinoma, etc. In particular, the phenotypic inclusion criterion can then also comprise a stage or a degree of the corresponding tumor disease. In particular, the at least one relevant region can then be encompassed by a genome of the corresponding tumor.

The inventors have recognized that the described method is suitable in particular for creating a gene panel plan in relation to tumor diseases.

At least one embodiment of the invention optionally relates to a computer-implemented training method for providing a trained function. The training method comprises a method step of receiving an available set of clinical trials and patient data of a patient. The training method further comprises a method step of receiving clinical trials relevant to the patient. The clinical trials relevant to the patient and the available set of clinical trials as well as the patient data are interrelated in this case. The available set of clinical trials in this case comprises the clinical trials relevant to the patient. The training method further comprises a method step of training a function based on the available set of clinical trials, the patient data and the clinical trials relevant to the patient. The training method further comprises a method step of providing the trained function.

In particular, the clinical trials relevant to the patient for training the trained function were determined manually from the available set of clinical trials.

At least one embodiment of the invention further relates to a determination system for creating an individual gene panel plan comprising an interface and a computing unit. In this case the interface and/or the computing unit are/is embodied to receive and/or determine a plurality of clinical trials. Each clinical trial of the plurality of clinical trials in this case comprises a molecular genetic inclusion criterion. The molecular genetic inclusion criterion in this case relates to gene information relevant to the respective clinical trial. In this case the computing unit is further embodied for determining, for each clinical trial of the plurality of clinical trials, at least one genomic region to which the gene information of the clinical trial relates. In this case the computing unit is further embodied to create a gene panel plan based on the genomic regions determined in respect of the plurality of clinical trials. In this case the interface is further embodied to provide the gene panel plan.

Such a determination system may be embodied in particular to perform the above-described method for creating an individual gene panel plan and its embodiments. The determination system is embodied to perform this method and its aspects in that the interface and the computing unit are embodied to perform the corresponding method steps.

In particular, the interface may comprise more than one subsidiary interface. In particular, the computing unit may comprise more than one subsidiary computing unit.

At least one embodiment of the invention also relates to a computer program product comprising a computer program, as well as to a computer-readable medium. A largely software-based implementation has the advantage that determination systems already used previously can also be easily upgraded via a software update in order to operate in the manner described. In addition to the computer program, such a computer program product may, where applicable, comprise additional constituent parts such as e.g. a set of documentation and/or additional components, as well as hardware components, such as e.g. hardware keys (dongles, etc.) to enable use of the software.

In particular, at least one embodiment of the invention also relates to a computer program product comprising a computer program which can be loaded directly into a memory of a determination system and having program sections for performing all steps of the above-described method for creating an individual gene panel plan and its embodiments when the program sections are executed by the determination system.

In particular, at least one embodiment of the invention relates to a computer-readable storage medium on which are stored program sections that can be read and executed by a determination system in order to perform all steps of at least one embodiment of the above-described method for creating an individual gene panel plan and its embodiments when the program sections are executed by the determination system.

At least one embodiment of the invention optionally relates to a training system for providing a trained function comprising a training interface and a training computing unit. The training interface is embodied to receive an available set of clinical trials and patient data of a patient. The training interface is further embodied to receive clinical trials relevant to the patient. In this case the clinical trials relevant to the patient and the available set of clinical trials as well as the patient data are interrelated. In this case the available set of clinical trials comprises the clinical trials relevant to the patient. The training computing unit is embodied to train a function based on the available set of clinical trials, the patient data and the clinical trials relevant to the patient. The training interface is further embodied to provide the trained function.

At least one embodiment of the invention optionally relates also to a computer program product comprising a computer program, as well as to a computer-readable medium. A largely software-based implementation has the advantage that training systems already used previously can also be easily upgraded via a software update in order to operate in the manner described. In addition to the computer program, such a computer program product may, where applicable, comprise additional constituent parts such as e.g. a set of documentation and/or additional components, as well as hardware components, such as e.g. hardware keys (dongles, etc.) to enable use of the software.

In particular, at least one embodiment of the invention optionally relates also to a computer program product comprising a computer program which can be loaded directly into a memory of a training system and having program sections for performing all steps of at least one embodiment of the above-described training method for providing a trained function and its aspects when the program sections are executed by the training system.

In particular, at least one embodiment of the invention optionally relates to a computer-readable storage medium on which are stored program sections that can be read and executed by a training system in order to perform all steps of at least one embodiment of the above-described training method for creating an individual gene panel plan and its aspects when the program sections are executed by the training system.

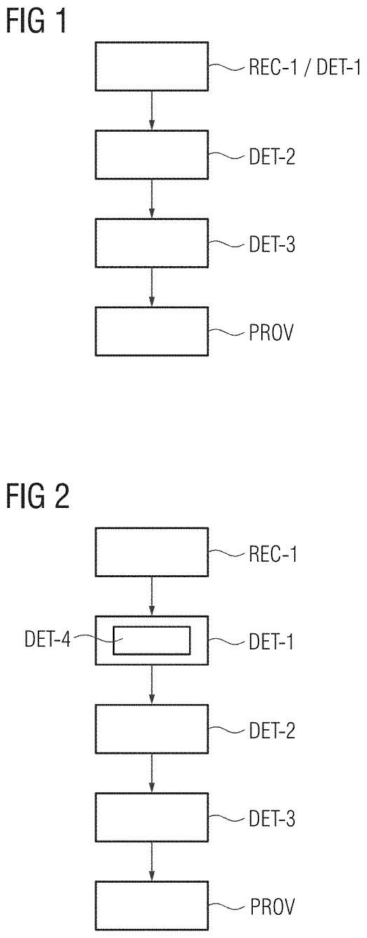

FIG. 1 shows a first example embodiment of a method for creating an individual gene panel plan.

In a gene panel, individual genes and/or genome regions are analyzed. It can be established during this process whether the genes and/or genome regions possess certain expressions or characteristics. Which genes and/or genome regions are analyzed in a gene panel can be defined or specified in a gene panel plan. A gene panel plan of the type can be created individually for a patient via the method described in the following.

In a method step of receiving REC-1 and/or determining DET-1 a plurality of clinical trials, the plurality of clinical trials is received and/or determined. In particular, the plurality of clinical trials can be received via an interface SYS.IF of a determination system SYS.

In the method step of receiving REC-1 the plurality of clinical trials, the clinical trials are received in particular by one of the following databases: ClinicalTrials.gov (https://clinicaltrials.gov/), International Clinical Trials Registry Platform (acronym: ICTRP) (https://www.who.int/clinical-trials-registry-platform), Deutsches Register Klinischer Studien (acronym: DRKS; German Clinical Trials Register) (https://www.drks.de/drks_web/) and/or EU Clinical Trials Register (https://www.clinicaltrialsregister.eu/). In particular, a selection of clinical trials of the set of clinical trials available on the corresponding database can be received. In other words, the plurality of clinical trials can comprise the selection of the available set of clinical trials.

In the method step of determining DET-1 the plurality of clinical trials, the plurality of clinical trials can be determined for example from the available set of clinical trials. In this case the plurality of clinical trials can be determined via a computing unit SYS.CU of the determination system SYS.

A clinical trial is in particular designed for treating a disease affecting a limited number of trial participants or patients. The treatment may in particular comprise testing a drug and/or a therapy. In particular, at least one of the clinical trials of the plurality of clinical trials may be designed for treating a tumor disease.

In an actual example embodiment, the plurality of clinical trials may be designed for early detection of breast cancer or breast carcinomas. The treatment of breast carcinomas can be optimized as a result of the early detection.

In particular, the clinical trial may be designed for a specific group of patients that have at least one characteristic in common. A common characteristic of the type can be mapped for example via a molecular genetic inclusion criterion. In other words, the molecular genetic inclusion criterion comprises a condition that a patient must satisfy in order to be eligible to take part in the clinical trial. In this case each clinical trial of the plurality of clinical trials comprises a molecular genetic inclusion criterion. The molecular genetic inclusion criterion of a clinical trial relates in this case to gene information relevant to the respective clinical trial. The gene information can in this case define in particular the common characteristic of the group of patients that are eligible to take part in the corresponding clinical trial. In particular, the gene information relates to a genetic characteristic of the group of patients. In other words, the gene information relates to information about the human genome. Alternatively, the gene information may relate to information about an animal genome. The gene information may relate to at least one region of a genome relevant to the clinical trial. In particular, a condition for participation in the clinical trial may be that the relevant region of the genome has a certain expression. In particular, the gene information may comprise this condition. The at least one relevant region may comprise a subregion of a gene. Alternatively, the at least one relevant region may comprise the entire gene. In particular, the at least one relevant region may contain a gene mutation. In particular, the gene information may then comprise a name of the region containing the gene mutation and/or a name for the gene mutation. In other words, the gene information may indirectly define at least one relevant region in this way. If the relevant region contains no gene mutation, the gene information can define the relevant region indirectly via a name of the relevant region. Alternatively or in addition, the gene information may comprise coordinates of the at least one relevant region. In this way, the gene information can directly define the at least one relevant region.

In the actual example embodiment, one clinical trial of the plurality of clinical trials comprises, as the molecular inclusion criterion, the following gene information: “BRCA1”. In other words, this means that patients carrying a mutation of the BRCA1 gene are eligible to take part in the clinical trial. Another clinical trial of the plurality of clinical trials comprises the gene information “BRCA1p1” in the actual example embodiment. In other words, patients carrying a mutation in the corresponding subregion of the BRCA1 gene are eligible to take part in the clinical trial.

In a method step of determining DET-2 at least one genomic region, at least one genomic region to which the gene information relates is determined for each of the clinical trials of the plurality of clinical trials. In this case the at least one genomic region can be determined for each of the clinical trials via the computing unit SYS.CU of the determination system SYS.

The at least one genomic region in this case describes the at least one relevant region, defined in the gene information, for each clinical trial of the plurality of clinical trials. The at least one genomic region in this case describes the at least one relevant region based upon coordinates of the relevant region on the genome or of the relevant subregion of the gene on the genome. In particular, parts of a gene on the genome can also be localized via the coordinates. If the at least one relevant region comprises a gene mutation, the exact location of the gene mutation on the gene can be specified via the coordinates. The coordinates encompassed by the genomic region can be copied directly from the gene information in the method step of determining DET-2 the at least one genomic region if the gene information includes the coordinates. Alternatively, the coordinates can be derived from the name included in the gene information in the method step of determining DET-2 the at least one genomic region. The coordinates can be derived in particular from the name of the at least one relevant region via a database. Examples of such a database are the ENSEMBL database (https://grch37.ensembl.org/index.html) or the NCBI database (https://www.ncbi.nlm.nih.gov/). In particular, the name of the at least one relevant region can also be derived from the name of the gene mutation encompassed by the relevant region. The name of a gene mutation may be the KRAS mutation, for example. In this case the name of the relevant region encompassing the gene mutation is the KRAS gene. The relevant region corresponds in this example to an entire gene. In another example, the name of the relevant region may be “EGFR 21 exon”. In this case the name defines a part, in particular the Exon 21 of a gene. The associated gene mutation “EGFR exon 21 deletion” corresponds in this case to a mutation of the gene at the site of the Exon 21.

In the actual example embodiment, the coordinates of the BRCA1 gene determined via one of the databases read “Chromosome 17: 41,196,312-41,322,262” in CRCh37 coordinates and the coordinates of the subregion BRCA1p1 “Chromosome 17: 41,320,187-41,320,266” in GRCh37 coordinates. The corresponding genomic regions of the two clinical trials are therefore described via the coordinates.

The at least one genomic region of a clinical trial of the plurality of clinical trials may in particular comprise a buffer zone around the coordinates of the at least one relevant region. The buffer zone can likewise be defined on the genome via coordinates. The buffer zone can be embodied in such a way that it can be ensured that the relevant region is located within the genomic region defined by the coordinates. In particular, it can be ensured in this way that even in the event of inaccuracies in an extracting of the genomic region from a genome for the gene panel, the extracted genomic region comprises the relevant region. In particular, the buffer zone can be embodied for example in such a way that the genomic region always comprises an entire gene, even if the relevant region comprises only a subregion of a gene. Alternatively or in addition, the buffer zone can be embodied in such a way that in each case the genes adjoining the gene encompassing the relevant region are encompassed by the buffer zone.

In the actual example embodiment, the buffer zone for the BRCA1 gene can comprise the complete segment 41 on the 17th chromosome (Chromosome 17: 41).

In a method step of creating DET-3 the gene panel plan, the gene panel plan is created based on the genomic regions determined in respect of the plurality of clinical trials. In this case the gene panel plan can be created via the computing unit SYS.CU of the determination system SYS.

In particular, the gene panel plan comprises all the genomic regions of the plurality of clinical trials. In particular, the gene panel plan comprises the coordinates of the genomic regions.

In a method step of providing PROV the gene panel plan, the gene panel plan is provided. In this case the gene panel plan can be provided via the interface SYS.IF of the determination system SYS.

In particular, the gene panel plan is provided to a user. The gene panel plan can be provided to the user in the form of a .bed document. In particular, the user may be a provider of gene panels. In particular, the user can produce a gene panel based on the gene panel plan and analyze the genomic regions included in the gene panel plan via the gene panel. In other words, the gene panel comprises the genomic regions of the patient defined in the gene panel plan for the analysis. In particular, the genomic regions of a genome of the patient that are defined by the gene information are analyzed. In particular, the genome is obtained from a tumor cell if the corresponding clinical trial is designed for a treatment of a tumor disease.

FIG. 2 shows a second example embodiment of a method for creating an individual gene panel plan.

The method steps of receiving REC-1 and/or determining DET-1 a plurality of clinical trials, of determining DET-2 at least one genomic region for each of the clinical trials of the plurality of clinical trials, of creating DET-3 the gene panel plan and of providing PROV the gene panel plan are embodied according to the description referring to FIG. 1.

The method step of determining DET-1 the plurality of clinical trials comprises a method step of determining DET-4 clinical trials relevant to a patient from an available set of clinical trials by filtering the available set of clinical trials. In this case the plurality of clinical trials comprises the trials relevant to the patient. In this case the trials relevant to a patient can be determined via the computing unit SYS.CU of the determination system SYS.

In the method step of receiving DET-1 the plurality of clinical trials, in particular the available set of clinical trials comprising the plurality of clinical trials can be received. The available set of clinical trials can in this case be received in particular by one of the above-cited databases.

In the method step of determining DET-4 clinical trials relevant to the patient, the clinical trials are determined from the available set of clinical trials which are relevant to the patient. In particular, the available set of clinical trials can be filtered for this purpose. For this purpose, at least one clinical trial of the plurality of clinical trials may comprise a phenotypic inclusion criterion. The available set of clinical trials can then be filtered based upon the phenotypic inclusion criterion. The phenotypic inclusion criterion may comprise at least one of the following criteria: an age of the trial participants, a place of residence of the trial participants, a disease of the trial participants, a stage in the disease of the trial participants. Via the phenotypic inclusion criterion it is therefore possible to define, in addition to the molecular genetic inclusion criterion, at least one further common characteristic of the group of patients that are eligible to take part in the corresponding clinical trial.

Via the age of the trial participants, it can be defined for example which age a patient must be in order to be able to take part in the corresponding clinical trial or to be eligible to take part. For example, the phenotypic inclusion criterion may specify that only patients aged between 18 and 60 years old are allowed to take part in the clinical trial. Many possible age restrictions for participation are possible in this way.

Via the place of residence, the phenotypic inclusion criterion can localize a geographical region in which a patient wishing to take part in the corresponding clinical trial should live. In this way it can be ensured for example that the patient lives in a geographical region in which the clinical trial is authorized. Alternatively or in addition, it can be ensured that the patient is able to travel for regular checkups and/or follow-up examinations. For example, a patient living in the USA cannot take part, or can take part only with special permission, in a clinical trial that is only approved for Germany. Furthermore, a necessary check on a state of health of the patient is possibly not provided due to the great distance.

Via a definition of the disease, the phenotypic inclusion criterion can narrow down the group of patients to the patients carrying the disease for which the corresponding clinical trial is designed. For example, a novel chemotherapy for treating a lung carcinoma can be tested in a clinical trial. In this case only patients suffering from a corresponding lung carcinoma are eligible to take part in the clinical trial. The phenotypic inclusion criterion can also localize an expression of the disease, for example. For example, the location of the lung carcinoma can be encompassed by the phenotypic inclusion criterion.

The phenotypic inclusion criterion may also specify the stage of the disease for participation in the clinical trial. The stage of the disease is to be understood as synonymous with a degree of the disease. In particular, only patients whose disease has not yet reached a certain stage, or whose disease has already exceeded a certain stage, may take part in the corresponding trial. For example, the international union against cancer (Union for International Cancer Control, acronym: UICC) has specified various stages for breast cancer which can serve as phenotypic inclusion criteria.

Further possible phenotypic inclusion criteria are conceivable, such as, for example, gender, preexisting conditions, previous treatments, etc.

For the filtering process, patient data of the patient for whom the relevant trials are to be determined can be received. In this case the patient data can be contained in particular in a patient's electronic health record. Alternatively, the patient data can be provided by a treating physician or by the patient or by an assistant. The patient data can in this case comprise information about the age of the patient, the place of residence of the patient, the disease of the patient, the stage in the disease of the patient, a preexisting condition of the patient, etc. After the filtering, the plurality of clinical trials can comprise the clinical trials for which the patient satisfies at least one phenotypic inclusion criterion. In particular, the plurality of clinical trials can comprise the clinical trials for which the patient satisfies all the phenotypic inclusion criteria. In other words, the clinical trials of the available set of clinical trials can be classified as relevant to the patient for which the patient satisfies at least one or all of the phenotypic inclusion criteria.

Clinical trials of the available set of clinical trials that comprise no phenotypic inclusion criterion can be assigned by default to the plurality of clinical trials. Alternatively, the clinical trials cannot be assigned by default to the plurality of clinical trials.

In particular, the plurality of clinical trials then comprises only clinical trials that are relevant to the patient. In particular, the plurality of clinical trials may also comprise clinical trials for which it is not known whether they could be relevant to the patient.

In the actual example embodiment described in FIG. 1, the patient is a 35-year-old female patient for whom relevant clinical trials in respect of early detection of breast cancer are to be determined. Based upon the patient data, all clinical trials are determined from the available set of clinical trials that comprise the following phenotypic inclusion criteria: “gender”: female, “age”: 30-50, “disease”: breast cancer. The treating physician provides the corresponding patient data for this purpose. The clinical trials determined in this way describe the clinical trials relevant to the patient.

In an alternative example embodiment to the illustrated example embodiment, the method step of determining DET-4 clinical trials relevant to a patient can be performed after the method step of determining DET-2 at least one genomic region for each clinical trial of the plurality of clinical trials. In particular, at least one genomic region can then be determined for all clinical trials of the available set of clinical trials in the method step of determining DET-2 at least one genomic region.

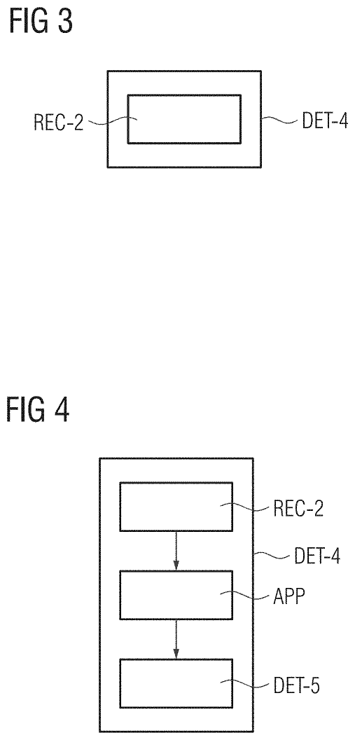

FIG. 3 shows a first example embodiment of a method step for determining DET-4 clinical trials relevant to a patient.

The method steps of receiving REC-1 and/or determining DET-1 a plurality of clinical trials, of determining DET-2 at least one genomic region for each of the clinical trials of the plurality of clinical trials, of creating DET-3 the gene panel plan and of providing PROV the gene panel plan are embodied according to the description referring to FIG. 1.

In this example embodiment, at least one clinical trial of the plurality of clinical trials can comprise at least one phenotypic inclusion criterion as described in the description referring to FIG. 2.

The method step of determining DET-4 clinical trials relevant to the patient comprises a method step of receiving REC-2 patient data. In this case the patient data can be received via the interface SYS.IF of the determination system SYS.

In the method step of receiving REC-2 the patient data, the patient data of the patient is received. In this case the patient data can be embodied as described above. The patient data can be received in this case in particular in the form of a patient's electronic health record. Alternatively, the patient data can be received in the form of a user input.

In the method step of determining DET-4 trials relevant to the patient, the filtering is based on a synchronizing of the phenotypic inclusion criterion with the patient data. In particular, the clinical trial which comprises an inclusion criterion that the patient satisfies based upon the patient data can be classified as a relevant trial. For example, it can be checked based upon the patient data whether the patient suffers from the disease which is a prerequisite for taking part in a clinical trial based upon the phenotypic inclusion criterion.

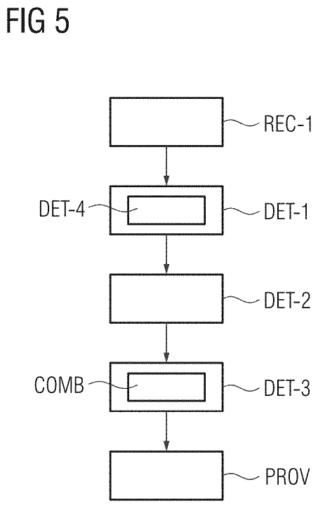

FIG. 4 shows a second example embodiment of a method step for determining DET-4 clinical trials relevant to a patient.

The method steps of receiving REC-1 and/or determining DET-1 a plurality of clinical trials, of determining DET-2 at least one genomic region for each of the clinical trials of the plurality of clinical trials, of creating DET-3 the gene panel plan and of providing PROV the gene panel plan are embodied according to the description referring to FIG. 1.

In this example embodiment, at least one clinical trial of the plurality of clinical trials can comprise at least one phenotypic inclusion criterion as described in the description referring to FIG. 2.

The method step of determining DET-4 clinical trials relevant to the patient comprises a method step of receiving REC-2 patient data, a method step of applying APP a trained function and a method step of determining DET-5 clinical trials relevant to the patient based on a relevance parameter.

In the method step of receiving REC-2 the patient data, the patient data of the patient is received. In this case the patient data can be received via the interface SYS.IF of the determination system SYS. The patient data can in this case be embodied as described above.

In the method step of applying APP the trained function, the trained function is applied to the available set of clinical trials and the patient data. In this case the trained function can be applied to the available set of clinical trials and the patient data via the computing unit SYS.CU of the determination system SYS. In this case a relevance parameter is determined for each clinical trial of the available set of clinical trials. The relevance parameter specifies a relevance of the corresponding clinical trial to the patient. In other words, by applying APP the trained function it is possible to classify the clinical trials of the available set of clinical trials in terms of their relevance to the patient. For example, the classification can be conducted into classes between “0” and “3”. In this case, “0” can mean that the correspondingly classified clinical trial is not relevant to the patient, “3” can mean that the correspondingly classified clinical trial is very relevant to the patient. The classes “1” and “2” can in this case denote a gradation between “0” and “3”. Alternative classifications and gradation units are possible.

In the method step of determining DET-5 clinical trials relevant to the patient based on the relevance parameter, the clinical trials relevant to the patient are determined from the available set of clinical trials. In particular, this method step can be performed via the computing unit SYS.CU of the determination system SYS. In particular, in the case of the above-cited example in respect of the classification, the clinical trials having a relevance parameter of “3” or “2” can be added to the clinical trials relevant to the patient. In particular, the clinical trials can then be included in the plurality of clinical trials.

FIG. 5 shows a third example embodiment of a method for creating an individual gene panel plan.

The method steps of receiving REC-1 and/or determining DET-1 a plurality of clinical trials, of determining DET-2 at least one genomic region for each of the clinical trials of the plurality of clinical trials, of creating DET-3 the gene panel plan and of providing PROV the gene panel plan are embodied according to the description referring to FIG. 1. The method step of determining DET-4 clinical trials relevant to the patient can be embodied according to the description referring to FIG. 2 and/or to FIG. 3 and/or to FIG. 4.

The method step of creating DET-3 the gene panel plan comprises a method step of combining the genomic regions of the plurality of clinical trials to form at least one combined genomic region. In this case the gene panel plan comprises the at least one combined genomic region.

The at least one combined genomic region in this case comprises all the genomic regions of the plurality of clinical trials. Furthermore, the combined genomic region comprises no region on the genome that is not encompassed by at least one genomic region of the plurality of clinical trials.

The combined genomic region comprises a mathematical union of the genomic regions of all the clinical trials of the plurality of clinical trials. The at least one combined genomic region in this case comprises overlapping areas of different genomic regions once only. Regions on the genome that are encompassed by none of the genomic regions are also not encompassed by the at least one combined genomic region. In particular, the gene panel plan may comprise more than one combined genomic region.

In other words, at least one region on the genome that is encompassed by at least one genomic region is defined in the gene panel plan. The at least one region is referred to as at least one combined genomic region. The at least one combined genomic region may be defined on the genome in particular via coordinates.

In the actual example embodiment described in FIG. 1, the genomic regions of the two clinical trials of the plurality of clinical trials are: “Chromosome 17: 41,196,312-41,322,262” and “Chromosome 17: 41, 320,187-41,320,266”. Since the genomic region “Chromosome 17: 41,196,312-41,322,262” comprises the genomic region “Chromosome 17: 41, 320,187-41,320,266”, the combined genomic region is “Chromosome 17: 41,196,312-41,322,262”. If the above-described buffer zone is taken into account, the combined genomic region that comprises the two genomic regions of the clinical trials is “Chromosome 17: 41”.

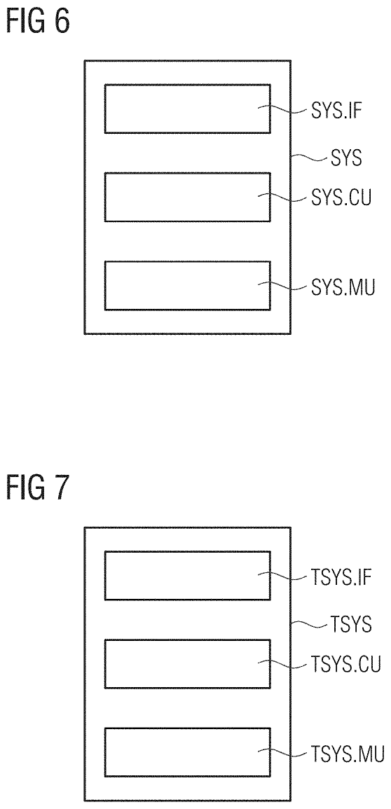

FIG. 6 shows a determination system SYS for creating an individual gene panel plan, FIG. 7 shows a training system TSYS for providing a trained function.

The illustrated determination system SYS for creating an individual gene panel plan is embodied to perform an inventive method for creating an individual gene panel plan. The illustrated training system TSYS is embodied to perform an inventive method for providing the trained function. The determination system SYS comprises an interface SYS.IF, a computing unit SYS.CU and a memory unit SYS.MU. The training system TSYS comprises a training interface TSYS.IF, a training computing unit TSYS.CU and a training memory unit TSYS.MU.

The determination system SYS and/or the training system TSYS may in particular be a computer, a microcontroller or an integrated circuit (IC). Alternatively, the system SYS and/or the training system TSYS may be a real or virtual computer network (a technical term for a real computer network is “cluster”, a technical term for a virtual computer network is “cloud”). The determination system SYS and/or the training system TSYS may be embodied as a virtual system that is implemented on a computer or a real computer network or a virtual computer network (a technical term is “virtualization”).

The interface SYS.IF and/or the training interface TSYS.IF may be a hardware or software interface (e.g. a PCI bus, USB or Firewire). The computing unit SYS.CU and/or the training computing unit TSYS.CU may comprise hardware and/or software components, for example a microprocessor or a device known as an FPGA (Field Programmable Gate Array). The memory unit SYS.MU and/or the training memory unit TSYS.MU may be embodied as a volatile working memory known as RAM (Random Access Memory) or as a nonvolatile mass storage device (hard disk drive, USB stick, SD card, solid state disk (SSD)).

The interface SYS.IF and/or the training interface TSYS.IF may in particular comprise a plurality of subsidiary interfaces that perform different method steps of the respective inventive method. In other words, the interface SYS.IF and/or the training interface TSYS.IF may be embodied as a plurality of interfaces SYS.IF and/or training interfaces TSYS.IF. The computing unit SYS.CU and/or the training computing unit TSYS.CU may in particular comprise a plurality of subsidiary computing units that perform different method steps of the respective inventive method. In other words, the computing unit SYS.CU and/or the training computing unit TSYS.CU may be embodied as a plurality of computing units SYS.CU and/or training computing units TSYS.CU.

Where not yet explicitly realized, though beneficial and within the meaning of the invention, individual example embodiments and individual subordinate aspects or features thereof may be combined with one another or interchanged without leaving the scope of the present invention. Advantages of the invention that are described with reference to one example embodiment are also relevant, where applicable, to other example embodiments without being cited explicitly.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for creating an individual gene panel plan, the method comprising:
at least one of receiving and determining a plurality of clinical trials, each respective clinical trial of the plurality of clinical trials including a molecular genetic inclusion criterion, wherein the molecular genetic inclusion criterion relates to gene information relevant to the respective clinical trial; and
for each respective clinical trial of the plurality of clinical trials,
determining at least one genomic region to which the gene information of the clinical trial relates,
creating the gene panel plan based on the genomic regions determined in respect of the plurality of clinical trials, and
providing the gene panel plan created.

2. The method of claim 1, wherein the gene information of a respective clinical trial relates to at least one region of a genome relevant to the clinical trial.

3. The method of claim 2, wherein the at least one relevant region of the genome contains a gene mutation relevant to the respective clinical trial.

4. The method of claim 3, wherein the gene information includes a name of the region of the genome containing at least one of the gene mutation and a name for the gene mutation.

5. The method of claim 2, wherein the at least one genomic region comprises coordinates of the at least one region of the genome relevant to the clinical trial.

6. The method of claim 5, wherein the at least one genomic region also includes a buffer zone around the coordinates of the at least one relevant region of the genome.

7. The method of claim 1, wherein the determining of the plurality of clinical trials comprises:
determining clinical trials relevant to a patient from an available set of clinical trials by filtering the available set of clinical trials, wherein the plurality of clinical trials includes the clinical trials relevant to the patient.

8. The method of claim 7,
wherein at least one clinical trial of the plurality of clinical trials includes at least one phenotypic inclusion criterion,
wherein the clinical trial is designed for treating a disease affecting trial participants,
wherein the phenotypic inclusion criterion comprises at least one of:
an age of the trial participants, a place of residence of the trial participants, the disease affecting the trial participants, a stage in the disease affecting the trial participants, and
wherein the determining of the clinical trials relevant to a patient is based on the at least one phenotypic inclusion criterion.

9. The method of claim 8, wherein the determining of the clinical trials relevant to a patient from an available set of clinical trials comprises:
receiving patient data of the patient, wherein the filtering is based on a synchronizing of the phenotypic inclusion criterion and the patient data.

10. The method of claim 7, wherein the determining of the clinical trials relevant to a patient from an available set of clinical trials comprises:
receiving patient data of the patient;
applying a trained function to the available set of clinical trials and the patient data, wherein a relevance parameter is determined for each respective clinical trial of the available set of clinical trials; and
determining clinical trials relevant to the patient based on the relevance parameter.

11. The method of claim 1, wherein the creating of the gene panel plan comprises:
combining the genomic regions of the plurality of clinical trials to form at least one combined genomic region, wherein the gene panel plan includes the at least one combined genomic region.

12. The method of claim 11, wherein the at least one combined genomic region includes a union of all the genomic regions of the plurality of clinical trials.

13. The method of claim 1, wherein at least one clinical trial of the plurality of clinical trials is designed for treating a tumor disease.

14. A determination system for creating an individual gene panel plan, comprising:
an interface; and
a computing unit,
wherein at least one of the interface and the computing unit is embodied to at least one of receive and determine a plurality of clinical trials, each respective clinical trial of the plurality of clinical trials including a molecular genetic inclusion criterion, wherein the molecular genetic inclusion criterion relates to gene information relevant to the respective clinical trial;
wherein the computing unit is further embodied to determine, for each respective clinical trial of the plurality of clinical trials, at least one genomic region to which the gene information of the clinical trial relates,
wherein the computing unit is further embodied to create a gene panel plan based on the genomic regions determined in respect of the plurality of clinical trials, and
wherein the interface is further embodied to provide the gene panel plan created.

15. A non-transitory computer program product storing a computer program, directly loadable into a memory of a determination system and including program sections for performing the method of claim 1 when the program sections are executed by the determination system.

16. A non-transitory computer-readable storage medium storing program sections, readable and executable by a determination system, to perform the method of claim 1 when the program sections are executed by the determination system.

17. The method of claim 3, wherein the at least one genomic region comprises coordinates of the at least one region of the genome relevant to the clinical trial.

18. The method of claim 17, wherein the at least one genomic region also includes a buffer zone around the coordinates of the at least one relevant region of the genome.

19. The method of claim 2, wherein the determining of the plurality of clinical trials comprises:
determining clinical trials relevant to a patient from an available set of clinical trials by filtering the available set of clinical trials, wherein the plurality of clinical trials includes the clinical trials relevant to the patient.

20. The method of claim 19,
wherein at least one clinical trial of the plurality of clinical trials includes at least one phenotypic inclusion criterion,
wherein the clinical trial is designed for treating a disease affecting trial participants,
wherein the phenotypic inclusion criterion comprises at least one of:
an age of the trial participants, a place of residence of the trial participants, the disease affecting the trial participants, a stage in the disease affecting the trial participants, and
wherein the determining of the clinical trials relevant to a patient is based on the at least one phenotypic inclusion criterion.
